SEQUENCE LISTING (25 sequences)

SEQ ID NO: 1 - DNA, 18 bp, Artificial Sequence
  Primer
ggtcagcacgattcggag

SEQ ID NO: 2 - DNA, 19 bp, Artificial Sequence
  Primer
gccaacaccaagtagacgg

SEQ ID NO: 3 - DNA, 19 bp, Artificial Sequence
  Primer
agccaacaccaagtagacg

SEQ ID NO: 4 - DNA, 20 bp, Artificial Sequence
  Primer
gagctcggccgcgaagaaag

SEQ ID NO: 5 - DNA, 19 bp, Artificial Sequence
  Primer
gagctcggccgcgaagaaa

SEQ ID NO: 6 - DNA, 21 bp, Artificial Sequence
  Primer
cagctcagcggattcttcggt

SEQ ID NO: 7 - DNA, 21 bp, Artificial Sequence
  Primer
tcagcggattcttcggtcgtg

SEQ ID NO: 8 - DNA, 19 bp, Artificial Sequence
  Primer
cggattcttcggtcgtggt

SEQ ID NO: 9 - DNA, 21 bp, Artificial Sequence
  Primer
gcgcagccaacaccaagtaga

SEQ ID NO: 10 - DNA, 20 bp, Artificial Sequence
  Primer
caacaccaagtagacgggcg

SEQ ID NO: 11 - DNA, 19 bp, Artificial Sequence
  Primer
tctctgcgaccatccgcac

SEQ ID NO: 12 - DNA, 19 bp, Artificial Sequence
  Primer
cgcggatctctgcgaccat

SEQ ID NO: 13 - DNA, 19 bp, Artificial Sequence
  Primer
ccgaattgcgaagggcgaa

SEQ ID NO: 14 - DNA, 20 bp, Artificial Sequence
  Primer
ccgaattgcgaagggcgaac

SEQ ID NO: 15 - DNA, 19 bp, Artificial Sequence
  Primer
gcgtaagtgggtgcgccag

SEQ ID NO: 16 - DNA, 19 bp, Artificial Sequence
  Primer
cggagacggtgcgtaagtg

SEQ ID NO: 17 - DNA, 19 bp, Artificial Sequence
  Primer
gacggtgcgtaagtgggtg

SEQ ID NO: 18 - DNA, 20 bp, Artificial Sequence
  Primer
gtgggcagcgatcagtgagg

SEQ ID NO: 19 - DNA, 21 bp, Artificial Sequence
  Primer
ggttcatcgaggaggtacccg

SEQ ID NO: 20 - DNA, 21 bp, Artificial Sequence
  Primer
tcaggtggttcatcgaggagg

SEQ ID NO: 21 - DNA, 21 bp, Artificial Sequence
  Primer
aggtggttcatcgaggaggta

SEQ ID NO: 22 - DNA, 19 bp, Artificial Sequence
  Primer
acaccaagtagacgggcga

SEQ ID NO: 23 - DNA, 19 bp, Artificial Sequence
  Primer
agccaacaccaagtagacg

SEQ ID NO: 24 - DNA, 19 bp, Artificial Sequence
  Primer
cggagacggtgcgtaagtg

SEQ ID NO: 25 - DNA, 21 bp, Artificial Sequence
  Primer
ctcagcggattcttcggtcgt

(19) United States
(12) Patent Application Publication
(10) Pub. No.: US 2014/0147851 A1
(43) Pub. Date: May 29, 2014

(54) METHODS AND KITS FOR DETECTING CELL-FREE PATHOGEN-SPECIFIC NUCLEIC ACIDS

(71) Applicant: Mingwei Qian, Hockessin, DE (US)

(72) Inventor: Mingwei Qian, Hockessin, DE (US)

(73) Assignee: Occam Biolabs, Inc., Newark, DE (US)

(21) Appl. No.: 14/009,199

(22) PCT Filed: Apr. 2, 2012
(86) PCT No.: PCT/US12/31814

§ 371 Date: Jan. 14, 2014

Related U.S. Application Data
(60) Provisional application No. 61/470,774, filed on Apr. 1, 2011.

(51) Int. Cl. C12Q 1/68

(52) U.S. Cl. CPC C12Q 1/689
USPC 435/6.12

(57) ABSTRACT

The present invention relates to a method for detecting a target nucleic acid derived from a pathogen in a subject. The method comprises (a) amplifying the nucleic acid sequence of the target nucleic acid, which is obtained from a cell-free fraction of a blood sample from the subject, to produce a double stranded DNA is produced, and (b) detecting the double stranded DNA. The presence of the double stranded DNA indicates the presence of the target nucleic acid in the subject. Also provided are kits for detecting a target nucleic acid derived from a pathogen in a subject.

### CROSS-REFERENCE TO RELATED APPLICATION

This application claims the benefit of U.S. Provisional Application No. 61/470,774, filed Apr. 1, 2011, the contents of which are incorporated herein in their entireties for all purposes.

### FIELD OF THE INVENTION

The invention relates generally to methods and kits useful for detecting pathogen-specific nucleic acids in a subject.

### BACKGROUND OF THE INVENTION

Many pathogenic infections cause serious illness. Early detection of pathogens in individuals plays an important role in diagnosis and treatment of diseases or disorders known to be associated with such pathogens. Tuberculosis is a common infectious disease caused by various strains of mycobacteria, usually Mycobacterium tuberculosis. In many cases, it is lethal. Tuberculosis is diagnosed definitively by identifying Mycobacterium tuberculosis in a clinical sample (e.g., sputum or pus) by microbiological culturing the sample. An inconclusive diagnosis may be made using other tests such as radiology (e.g., chest x-rays), a tuberculin skin test, and an interferon Gamma Release Assays (IGRA).

Polymer chain reaction (PCR) technology has been used to detect Mycobacterium tuberculosis in samples, for example, sputum, urine, gastric aspirate, cerebrospinal fluid, pleural fluid, blood, and materials from abscesses, bone marrow, biopsy samples, resected tissues, or transbronchial biopsies, to provide early TB diagnosis. It has been reported that detection of TB DNA in a leukocyte fraction of peripheral blood from all 8 confirmed pulmonary TB patients in one study and 39 of 41 confirmed TB patients in another study. Schluger et al., Lancet 344:232-3 (1994); Cordos et al. Lancet 347:1082-5 (1996). However, these results were criticized by other researchers exploring blood-based PCR TB diagnosis. Kolk et al. Lancet., 344: 694 (1994); Palenque et al. Lancet. 344:1021 (1994); Aguado et al. Lancet. 347:1836-7 (1996). In the last two decades, tremendous efforts have been made to utilize “Blood TB PCR” assay for TB diagnostics, but with very limited success.

Most nucleic acids (e.g., DNA and RNA) in the body are located within cells, but a small amount of nucleic acids are found circulating freely in the plasma of individuals. These DNA and RNA molecules are believed to come from dying cells that release their contents into the blood as they break down.

Detection of a target RNA derived from a DNA pathogen may be used to differentiate active infection from latent infection. For example, detection of a target RNA derived from Mycobacterium tuberculosis (TB) may be used to differentiate active TB infection from latent TB infection and useful for TB diagnosis. Circulating nucleic acids (CNA) are DNA or RNA found in the bloodstream. Since the detection of fetus DNA from maternal peripheral blood, cell-free DNA and RNA from tumors, xenographs, transplants, and parasites have been found in host peripheral blood. CNA detection has been explored as a non-invasive diagnosis of a variety of clinical conditions. Unfortunately, it has not been successfully adopted for detecting pathogen-specific circulating nucleic acids with high sensitivity and high specificity.

Therefore, there remains a need for an early detection method for pathogens in individuals, for example, Mycobacterium tuberculosis, with high sensitivity and high specificity.

### SUMMARY OF THE INVENTION

The present invention relates to detection of cell-free pathogen-specific nucleic acids in a subject, and related detection kits.

According to one aspect of the present invention, a method for detecting a target nucleic acid derived from a pathogen in a subject is provided. The method comprises amplifying the nucleic acid sequence of the target nucleic acid, which is obtained from a cell-free fraction of a blood sample from the subject. A double stranded DNA is thereby produced. The method further comprises detecting the double stranded DNA. The presence of the double stranded DNA indicates the presence of the target nucleic acid in the subject. The cell-free fraction is preferably blood serum, blood plasma, pleural fluid, or CSF, more preferably blood serum or blood plasma.

The pathogen may be selected from the group consisting of bacteria, fungi and parasites. Preferably, the pathogen is Mycobacterium Tuberculosis (TB).

The target nucleic acid may be DNA or RNA. The nucleic acid sequence of the target nucleic acid may be derived from a DNA sequence of Mycobacterium Tuberculosis (TB) H37Rv, for example, selected from the group consisting of IS6110, IS1084, MPT 64, rrs, esat6, esat6-like, MDR, rpoB, katG, iniB and fragments thereof.

The double stranded DNA may have fewer than 100 bp, preferably 40-60 bp.

The blood sample from the subject may be in the amount of 0.2-10 ml, preferably 2-5 ml.

The nucleic acid sequence of the target nucleic acid may be amplified by polymer chain reaction (PCR), reverse transcription polymerase chain reaction (RT-PCR), transcription-mediated amplification (TMA), or ligase chain reaction (LCR). Preferably, the nucleic acid sequence is amplified by PCR.

The double stranded DNA may be detected by a detecting agent. The detecting agent may be a fluorescence labeled probe (e.g., a Taqman probe, Molecular beacon, or Scorpin), an intercalating fluorescence dye or a primer of Light Upon Extension (LUX). Preferably, the detecting agent is an intercalating fluorescence dye. The intercalating fluorescence dye may be selected from the group consisting of SYBR green, CytoGreen, Eva Green, BOXTO and SYTO9.

The method may further comprise concentrating the target nucleic acid in the cell-free fraction.

The method may further comprise preparing the cell-free fraction from the blood sample.

The method may further comprise diagnosing TB infection in the subject. The TB infection may be active or latent.

According to another aspect of the invention, a kit for detecting a target nucleic acid derived from a pathogen in a subject is provided. The kit comprises one or more reagents or materials for amplifying the nucleic acid sequence of the target nucleic acid, which may be DNA or RNA, obtained from a cell-free fraction of a blood sample from the subject to produce a double stranded DNA. The kit further comprises one or more reagents or materials for detecting the double stranded DNA. The pathogen may be selected from the group consisting of bacteria, fungi and parasites, preferably Mycobacterium Tuberculosis (TB). The nucleic acid sequence may be derived from a DNA sequence of Mycobacterium Tuberculosis (TB) H37Rv selected from the group consisting of IS6110, IS1084, MPT 64, rrs, esat6, esat6-like, MDR, rpoB, katG, iniB and fragments thereof.

The one or more reagents or materials for amplifying the target nucleic acid sequence may comprise a pair of primers, and the double stranded DNA may have 40-60 nucleotides. The pair of primers may have sequences of GGTCAGCACGATTCGGAG (SEQ ID NO: 1) and GCCAACACCAAGTAGACGG (SEQ ID NO: 2).

The one or more reagents or materials for detecting the double stranded DNA comprises a fluorescence labeled probe (e.g., a Taqman probe, Molecular beacon, or Scorpin), an intercalating fluorescence dye or a primer of Light Upon Extension (LUX), preferably an intercalating fluorescence dye. The intercalating fluorescence dye may be selected from the group consisting of SYBR green, CytoGreen, Eva Green, BOXTO and SYTO9.

### BRIEF DESCRIPTION OF THE DRAWINGS

FIG. 1 shows (A) amplification curves and (B) melting curves for short qPCR products using TB genomic DNA as templates.

FIG. 2 shows (A) amplification curves and (B) melting curves for short qPCR products for TB detection in plasma of monkeys.

FIG. 3 shows (A) amplification curves and (B) melting curves for short qPCR products for TB detection in human individuals using plasma fractions from 6 individuals clinically diagnosed with TB (TB, arrow A) or from 2 individuals not clinically diagnosed with TB (non-TB, arrow B).

FIG. 4 shows (A) amplification curves and (B) melting curves for short qPCR products for TB detection in a human individual clinically diagnosed with TB using a cell-free fraction of a pleural effusion sample from the individual (arrow A) and a sediment fraction of the same pleural effusion sample (arrow B).

FIG. 5 shows (A) amplification curves and (B) melting curves for short qPCR products for TB detection in two human individuals, A and B, who were clinically diagnosed with TB, using cell free fractions of plasma (PS) and CSF samples from each individual.

### DETAILED DESCRIPTION OF THE INVENTION

The present invention is based on the discovery of a novel nucleic acid amplification test (NAAT) for detecting target nucleic acids derived from pathogens such as Mycobacterium tuberculosis in a subject.

The present invention provides a method for detecting a target nucleic acid derived from a pathogen in a subject. The method comprises amplifying the nucleic acid sequence of the target nucleic acid, which is obtained from a cell-free fraction of a biological sample from the subject. A double stranded DNA is thereby produced. The method further comprises detecting the double stranded DNA. The presence of the double stranded DNA indicates the presence of the target nucleic acid in subject.

A subject may be an animal, including a mammal, for example, a human, a mouse, a cow, a horse, a chicken, a dog, a cat, and a rabbit. The animal may be an agricultural animal (e.g., horse, cow and chicken) or a pet (e.g., dog and cat). The subject is preferably a human or a mouse, more preferably a human. The subject may be a male or female. The subject may also be a newborn, child or adult. The subject may have suffered or predisposed to a disease or medical condition.

A pathogen may be selected from the group consisting of a bacterium, a parasite and a fungus. The bacterium may be Brucella, Treponema, Mycobacterium, Listeria, Legionella, Helicobacter, Streptococcus, Neisseria, Clostridium, Staphylococcus or Bacillus; and more preferably to Treponema pallidum, Mycobacterium tuberculosis, Mycobacterium leprae, Listeria monocytogenes, Legionella pneumophila, Helicobacter pylori, Streptococcus pneumoniae, Neisseria meningitis, Clostridium novyi, Clostridium botulinum, Staphylococcus aureus, and Bacillus anthracis, most preferably, Mycobacterium tuberculosis. The parasite may be Trichomonas, Toxoplasma, Giardia, Cryptosporidium, Plasmodium, Leishmania, Trypanosoma, Entamoeba, Schistosoma, Filariae, Ascaria, or Fasciola; and more preferably Trichomonas vaginalis, Toxoplasma gondii, Giardia intestinalis, Cryptosporidium parva, Plasmodium, Leishmania, Trypanosoma cruzi, Entamoeba histolytica, Schistosoma, Filariae, Ascaria, and Fasciola hepatica.

The term “nucleic acid” used herein refers to a polynucleotide comprising two or more nucleotides. It may be DNA or RNA. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, preferably at least about 90%, more preferably at least about 95%, identical to the nucleotide sequence of the original nucleic acid.

The term “derived from” used herein refers to an origin or source, and may include naturally occurring, recombinant, unpurified or purified molecules. A nucleic acid derived from an original nucleic acid may comprise the original nucleic acid, in part or in whole, and may be a fragment or variant of the original nucleic acid.

A “target nucleic acid” in the method according to the present invention is a nucleic acid, DNA or RNA, to be detected. A target nucleic acid derived from an organism is a polynucleotide that has a sequence derived from that of the organism and is specific to the organism. A target nucleic acid derived from a pathogen refers to a polynucleotide having a polynucleotide sequence derived from that specific the pathogen. For example, a target nucleic acid may be derived from Mycobacterium Tuberculosis (TB) H37Rv strain, and comprises a sequence specific to H37Rv strain. Examples of suitable TB H37Rv strain specific sequences include sequences of IS6110, IS1084, MPT 64, rrs, esat6, esat6-like, MDR, rpoB, katG, iniB, and fragments thereof. A target nucleic acid may be of any length, preferably having about 30-150 nucleotides, preferably about 40-100 nucleotides.

A biological sample may be any sample obtained from the subject. Examples of the biological samples include bodily fluid, cells and tissues. The bodily fluid may be blood serum or plasma, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites, or pericardial fluid. Preferably, the biological sample is a blood sample. The biological sample from the subject may be of any volume, for example, about 0.2-10 ml, preferably about 0.5-10 ml, more preferably about 2-10 ml, most preferably about 2-5 ml. The cell-free fraction is preferably blood serum, blood plasma, pleural fluid, or CSF, more preferably blood serum or blood plasma.

The term “cell-free fraction” of a biological sample used herein refers to a fraction of the biological sample that is substantially free of cells. The term “substantially free of cells” used herein refers to a preparation from the biological sample comprising fewer than about 20,000 cells per ml, preferably fewer than about 2,000 cells per ml, more preferably fewer than about 200 cells per ml, most preferably fewer than about 20 cells per ml. The cell-free fraction may be substantially free of host genomic DNA. Host genomic DNA are large pieces of DNA (e.g., longer than about 10, 20, 30, 40, 50, 100 or 200 kb) derived from the subject. For example, the cell-free fraction of a biological sample from a subject may comprise less than about 1,000 ng per ml, preferably less than about 100 ng per ml, more preferably less than about 10 ng per ml, most preferably less than about 1 ng per ml, of host genomic DNA.

The method of the present invention may further comprise preparing a cell-free fraction from a biological sample. The cell-free fraction may be prepared using conventional techniques known in the art. For example, a cell-free fraction of a blood sample may be obtained by centrifuging the blood sample for about 3-30 min, preferably about 3-15 min, more preferably about 3-10 min, most preferably about 3-5 min, at a low speed of about 200-20,000 g, preferably about 200-10,000 g, more preferably about 200-5,000 g, most preferably about 350-4,500 g. The biological sample may be obtained by ultrafiltration in order to separate the cells and their fragments from a cell-free fraction comprising soluble DNA or RNA. Conventionally, ultrafiltration is carried out using a 0.22 μm membrane filter.

The method of the present invention may further comprise concentrating (or enriching) the target nucleic acid in the cell-free fraction of the biological sample. The target nucleic acid may be concentrated using conventional techniques known in the art, such as solid phase absorption in the presence of a high salt concentration, organic extraction by phenol-chloroform followed by precipitation with ethanol or isopropyl alcohol, or direct precipitation in the presence of a high salt concentration or 70-80% ethanol or isopropyl alcohol. The concentrated target nucleic acid may be at least about 2, 5, 10, 20 or 100 times more concentrated than that in the cell-free fraction. The target nucleic acid, whether or not concentrated, may be used for amplification according to the method of the present invention.

The sequence of the target nucleic acid may be amplified to produce a double stranded DNA using various methods known in the art. For example, the sequence may be amplified by polymerase chain reaction (PCR), reverse transcription polymerase chain reaction (RT-PCR), transcription-mediated amplification (TMA), or ligase chain reaction (LCR). Preferably, the sequence of the target nucleic acid is amplified by quantitative real-time PCR (qPCR). A pair of primers may be designed to amplify a desirable sequence of the target nucleic acid to produce a double stranded DNA of a desirable length. For example, the pair of primers may have sequences of GGTCAGCACGATTCGGAG (SEQ ID NO: 1) and GCCAACACCAAGTAGACGG (SEQ ID NO: 2). The double stranded DNA may have fewer than about 100, 90, 80, 70, 60, 50, 40 or 30 nucleotides. For example, the double stranded DNA may have about 30-70 bp, preferably about 40-60 bp.

The double stranded DNA may be detected by various techniques known in the art. For example, the double stranded DNA may be detected by a detecting agent. The detecting agent may be selected from the group consisting of a fluorescence labeled probe (e.g., a Taqman probe, Molecular beacon, or Scorpin), an intercalating fluorescence dye, or a primer for Light Upon Extension (LUX). Preferably, the detecting agent is an intercalating fluorescence dye. The intercalating fluorescence dye may be SYBR green, CytoGreen, LC Green, Eva Green, BOXTO or SYTO9.

The method of the present invention may further comprise quantifying the copy number of the target nucleic acid in the subject. For example, the sequence of the target nucleic acid may be amplified by real time PCR (qPCR). A standard curve may be established for a standard nucleic acid with known number of copies and the detected fluorescence. Based on the standard curve, the copy number of a target nucleic acid may be determined based on the level of fluorescence after qPCR.

The method of the present invention may further comprise diagnosis of infection by the pathogen in the subject. For example, the pathogenic infection (e.g., TB infection) may be active or latent. Detection of RNA derived from a pathogen (e.g., a bacterium, a parasite or a fungus) may be used to differentiate active infection from latent infection. For example, detection of a target RNA derived from Mycobacterium tuberculosis (TB) may be used to differentiate active TB infection from latent TB infection, and thus contribute to diagnosis of active or latent TB infection. The method may provide a high sensitivity of, for example, at least about 50%, 60%, 70%, 80%, 90%, 95% or 99%, preferably at least about 80%, more preferably at least bout 90%, most preferably at least about 95%. The method may provide a high specificity of, for example, at least about 50%, 60%, 70%, 80%, 90%, 95% or 99%, preferably at least about 80%, more preferably at least bout 90%, most preferably at least about 95%.

For the detection methods of the present invention, various detection kits are provided. A kit for detecting a target nucleic acid derived from a pathogen in a subject is provided. The kit comprises (a) one or more reagents or materials for amplifying the nucleic acid sequence of the target nucleic acid obtained from a cell-free fraction of a biological sample from the subject to produce a double stranded DNA, and (b) one or more reagents or materials for detecting the double stranded DNA. The biological sample is preferably a blood sample.

In the kit of the present invention, the one or more amplifying reagents or materials may comprise a pair of primers suitable for producing a double stranded nucleic acid having fewer than about 100, 90, 80, 70, 60, 50, 40 or 30 nucleotides. The double stranded DNA may have about 30-70 base pairs (bp), preferably 40-60 bp. The primers may be designed to amplify a target sequence specific to the pathogen. The target sequence may be a sequence specific to Mycobacterium Tuberculosis (TB) H37Rv, for example, selected from the group consisting of IS6110, IS1084, MPT 64, rrs, esat6, esat6-like, MDR, rpoB, katG, iniB and fragments thereof. For example, The pair of primers may have sequences of GGTCAGCACGATTCGGAG (SEQ ID NO: 1) and GCCAACACCAAGTAGACGG (SEQ ID NO: 2).

In the kit of the present invention, the one or more detecting reagents or materials may comprise a detecting agent selected from the group consisting of a fluorescence labeled probe (e.g., a Taqman probe, Molecular beacon or Scorpin), an intercalating fluorescence dye, and a primer with LUX. Preferably, the detecting agent is an intercalating fluorescence dye. The intercalating fluorescence dye may be SYBR Green, CytoGreen, LC Green, Eva Green, BOXTO or SYTO9.

The kit of the present invention may further comprise one or more reagents or materials for preparing the cell-free fraction from the biological sample (e.g., blood sample) in an amount of, for example, about 0.2-10 ml, preferably about 0.5-10 ml, more preferably about 2-10 ml, most preferably about 2-5 ml. The cell-free fraction may be substantially free of cells comprising, for example, fewer than about 20,000 cells per ml, preferably fewer than about 2,000 cells per ml, more preferably fewer than about 200 cells per ml, most preferably fewer than about 20 cells per ml. The cell-free fraction may be substantially free of host genomic DNA. Host genomic DNA are large pieces of DNA (e.g., longer than about 10, 20, 30, 40, 50, 100 or 200 kb) derived from the subject. For example, the cell-free fraction of a biological sample from a subject may comprise less than about 1,000 ng per ml, preferably less than about 100 ng per ml, more preferably less than about 10 ng per ml, most preferably less than about 1.0 ng per ml, of host genomic DNA.

The kit of the present invention may further comprise one or more reagents or materials for isolating or purifying the target nucleic acid from the cell-free fraction. The target nucleic acid may be concentrated by at least about 2, 5, 10, 20 or 100 times more concentrated than that in the cell-free fraction. The target nucleic acid, whether or not concentrated, may be used for amplification according to the method of the present invention.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1

### Primer Design

The primer design program Primer3 (http://frodo.wi.mit.edu/) was used for the design of all primers for TB detection. To design primers specifically complementary to TB genomic DNA sequence, the complete genome of Mycobacterium tuberculosis H37Rv strain (GenBank Accession No. NC_{—}000962) was used as a reference. For primers specifically complementary to human genomic DNA, human genome was used as reference sequence from Gene Bank database.

Primers of a variety of amplicon sizes designed to amplify nucleic acids specific to TB H37rv strain were optimized using SYBR qPCR reaction followed by a melting curve analysis. They may be further validated by Agarose gel (3%) electrophoresis as evidenced by DNA bands of correct sizes without non-specific DNA products or primer-dimers. Exemplary TB primers are set forth in Table 1.

TABLE 1
Exemplary TB Primers

| Primers | SEQ ID NO: |
|---|---|
| GGTCAGCACGATTCGGAG | 1 |
| GCCAACACCAAGTAGACGG | 2 |
| AGCCAACACCAAGTAGACG | 3 |
| GAGCTCGGCCGCGAAGAAAG | 4 |
| GAGCTCGGCCGCGAAGAAA | 5 |
| CAGCTCAGCGGATTCTTCGGT | 6 |
| TCAGCGGATTCTTCGGTCGTG | 7 |
| CGGATTCTTCGGTCGTGGT | 8 |
| GCGCAGCCAACACCAAGTAGA | 9 |
| CAACACCAAGTAGACGGGCG | 10 |
| TCTCTGCGACCATCCGCAC | 11 |
| CGCGGATCTCTGCGACCAT | 12 |
| CCGAATTGCGAAGGGCGAA | 13 |
| CCGAATTGCGAAGGGCGAAC | 14 |
| GCGTAAGTGGGTGCGCCAG | 15 |
| CGGAGACGGTGCGTAAGTG | 16 |
| GACGGTGCGTAAGTGGGTG | 17 |
| GTGGGCAGCGATCAGTGAGG | 18 |
| GGTTCATCGAGGAGGTACCCG | 19 |
| TCAGGTGGTTCATCGAGGAGG | 20 |
| AGGTGGTTCATCGAGGAGGTA | 21 |
| ACACCAAGTAGACGGGCGA | 22 |
| AGCCAACACCAAGTAGACG | 23 |
| CGGAGACGGTGCGTAAGTG | 24 |
| CTCAGCGGATTCTTCGGTCGT | 25 |

### Example 2

### Real Time PCR (qPCR)

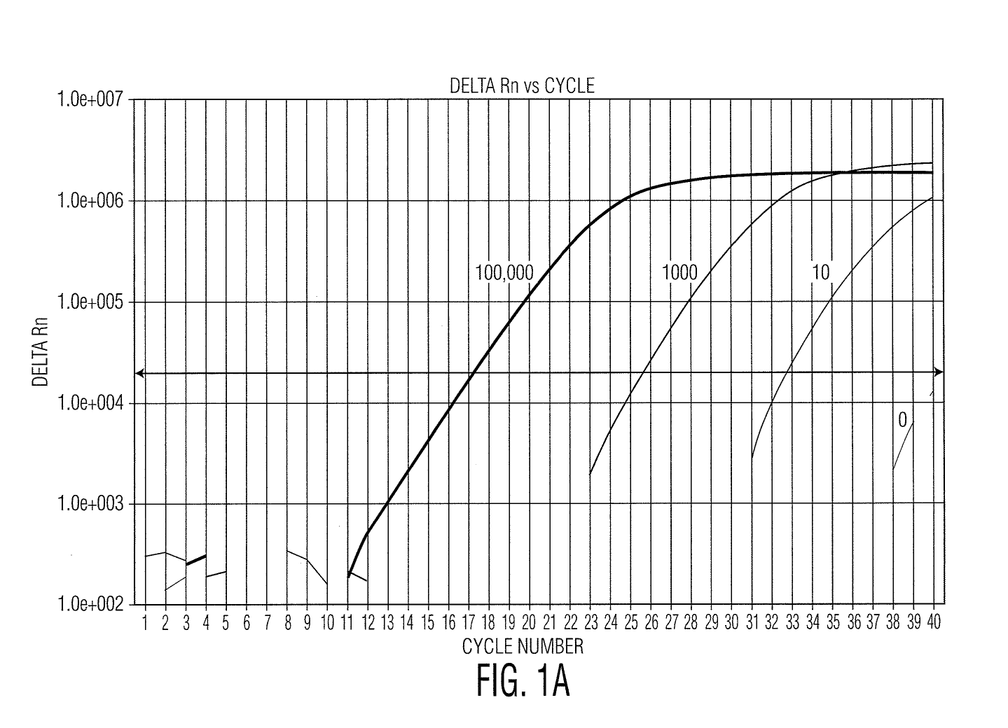

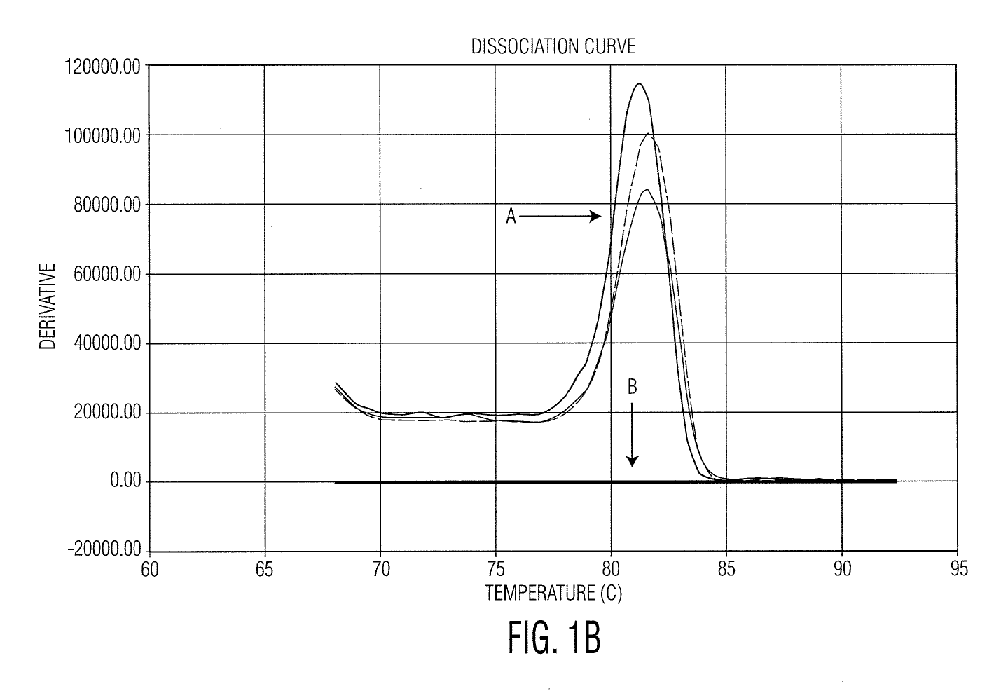

A serial of 10-fold dilutions of TB H37Rv genomic DNA were used as templates in real time qPCR reaction. A pair of primers having sequences of GGTCAGCACGATTCGGAG (SEQ ID NO: 1) and GCCAACACCAAGTAGACGG (SEQ ID NO: 2) was used to amplify a target sequence, an IS6110 insertion sequence, in the TB H37Rv genomic DNA. The PCR reaction program used included 95° C. 3 min, followed by 40 cycles of “94° C. 10 sec., 60° C. 10 sec. 72° C. 30 sec. with fluorescent detection” and a melting phase from 60° C. to 95° C. Amplification curves (FIG. 1A) generated for 1,000,000, 1,000 and 10 copies of the target nucleic acids showed increasing levels of accumulated fluorescence as the cycle number increased, and increasing threshold cycle (Ct) values as the copy number of the amplified sequence decreased. A standard curve of Ct values vs copy number could be generated based on the amplification curves, and useful for quantifying the copy number of any specific nucleic acid in a sample based on the accumulated fluorescence of the resulting qPCR products using a suitable pair of primers under the qPCR conditions. Melting curves (FIG. 1B) showed a specific peak for 1,000,000, 1,000 or 10 copies of the target nucleic acids (arrow A) and no specific peak when there was no template (i.e., 0 copy). There was no non-specific or primer-dimer noise peaks.

### Example 3

### TB Detection in Monkey Blood Specimens

In a preliminary experiment, a group of 6 Rhesus monkeys (Macaca mulatta) were inoculated with TB (Mycobacterium tuberculosis, stain H37Rv) at 50 CFU and 500 CFU/subject (2 animals for each infected group and two as control group). During the experiments, a tuberculin test (Tuberculin OT, Synbiotics Corp. CA), immunoassays for TB antibodies, release of cytokines, stimulated IFN-gamma were periodically performed. At the end of the experiment, samples were collected from the monkeys for pathological examinations and TB cultures. Whole blood samples were also collected biweekly.

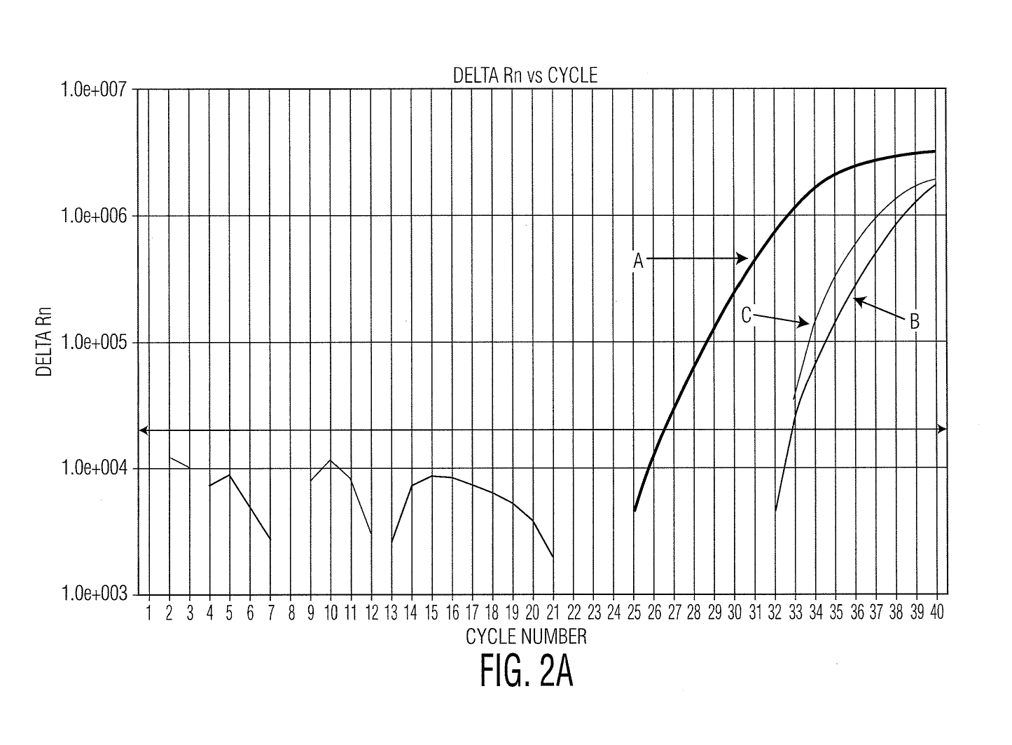

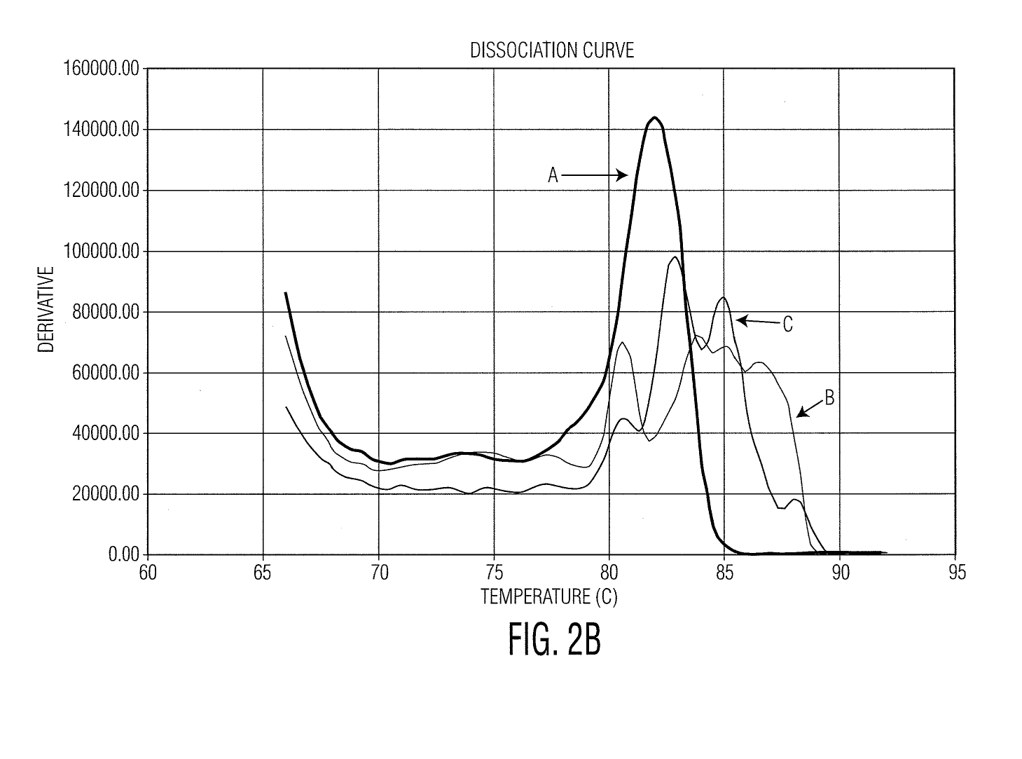

Fresh whole blood was collected after 6 and 8 weeks, and immediately centrifuged into 2 fractions, plasma and blood cells. Peripheral white blood cells (PWBC) were further isolated by Ficoll-Hypaque density gradient centrifugation (Sigma Chemical Co., Mo.). The separated fractions were immediately frozen at −80° C. These blood fractions were used for isolation of TB DNA for qPCR quantification. The TB DNA from the specimens were extracted with silica membrane centrifuge columns, E.Z.N.A.® Blood DNA Midi Kit (Omega Bio-tek, Inc., GA). The DNA extracted from whole blood, PWBC and plasma fractions were used as templates for qPCR quantification SYBR® Premix Ex Taq (Takara Bio USA, CA) following a qPCR protocol described in Example 2. The amplification curves (FIG. 2A) for plasma (A), PWBC (B) and whole blood (C) showed a much lower Ct value for plasma (A) than that for PWBC (B) or whole blood (C). The melting curves (FIG. 2B) showed a specific single peak for plasma (A) and several non-specific peaks for PWBC (B) and whole blood (C).

### Example 4

### TB Detection in Human Blood Specimens

Clinical samples (which were ready to be discarded after routine clinical lab tests) were collected from 92 individuals. Among them, 74 individuals were clinically diagnosed of TB, and 18 individuals were not clinically diagnosed for TB. Among these 18 individuals, 15 were diagnosed of other diseases.

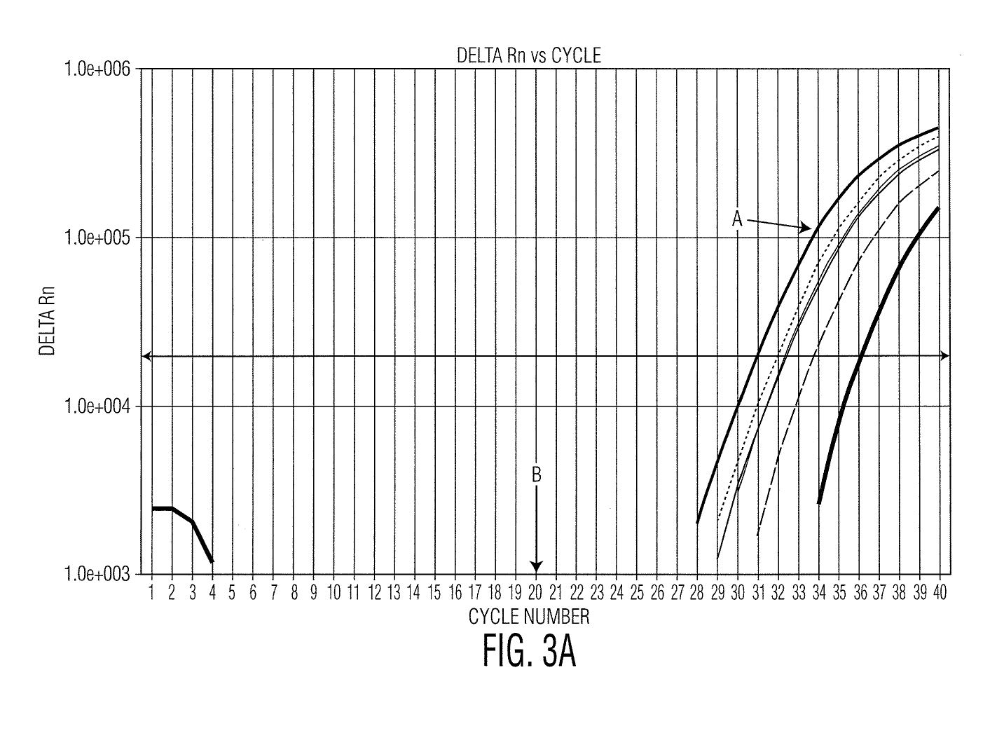

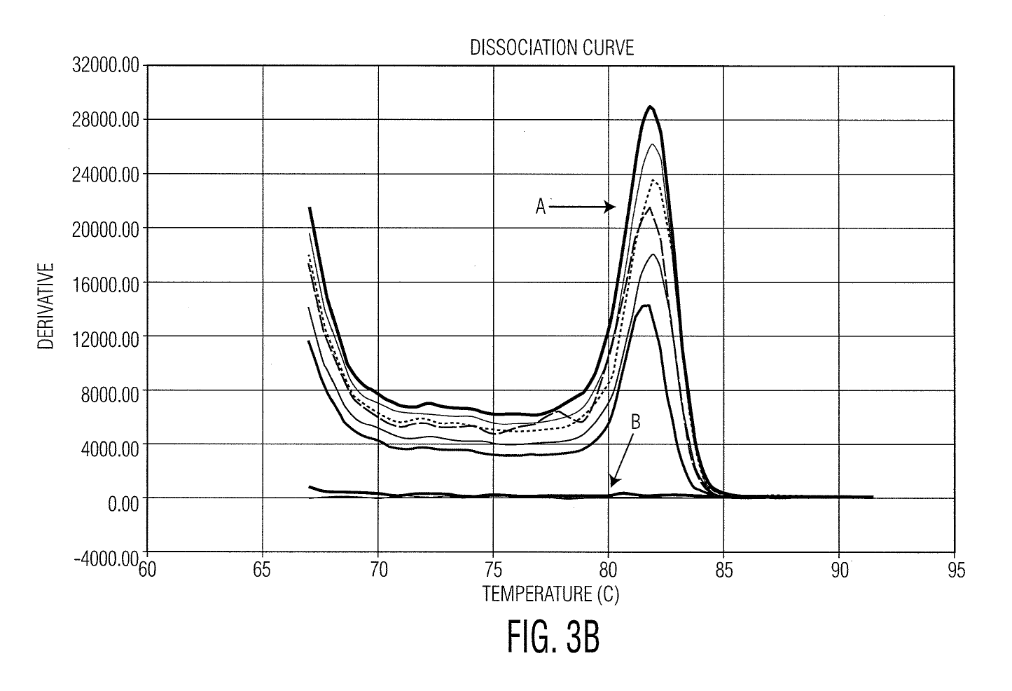

The clinical samples included blood samples, pleural effusion and cerebrospinal fluids (CSF). About 5 ml peripheral blood samples were collected into serum collection tubes or plasma collection tubes with anticoagulants EDTAK2. Both serum and plasma were separated by centrifugation at 1,600 g for 10 min. Serum and plasma aliquots were immediately frozen at −20° C. Pleural effusion and CSF were collected in tubes with or without anticoagulant EDTAK2, and separated into cell-free fractions and sediments after centrifugation at 5,000 g for 10 minutes. The cell-free fractions of blood plasma (PS), pleural effusion and CSF, and cellular fractions (the sediments) of the pleural effusion and CSF, were used for nucleic acid extraction, after lysis, denaturation, and Proteinase K digestion, with QIAamp Circulating Nucleic Acid Kit (Qiagen, CA). TB detection was carried out following the protocol described in Example 2. Amplification curves (FIG. 3A) and melting curves (FIG. 3B) for plasma (PS) fractions from 6 individuals clinically diagnosed of TB (TB plasma fractions, arrow A) and 2 individuals not clinically diagnosed for TB (non-TB plasma fractions, arrow B) show representative quantitative comparison. The TB specific short nucleic acid fragments of IS6110 (FIG. 3B) in the cell-free fractions of the blood samples were quantified using a standard curve described in Example 2 to have about 20-40 copies per ml of TB plasma fractions and 0 copy per ml of non-TB plasma fractions.

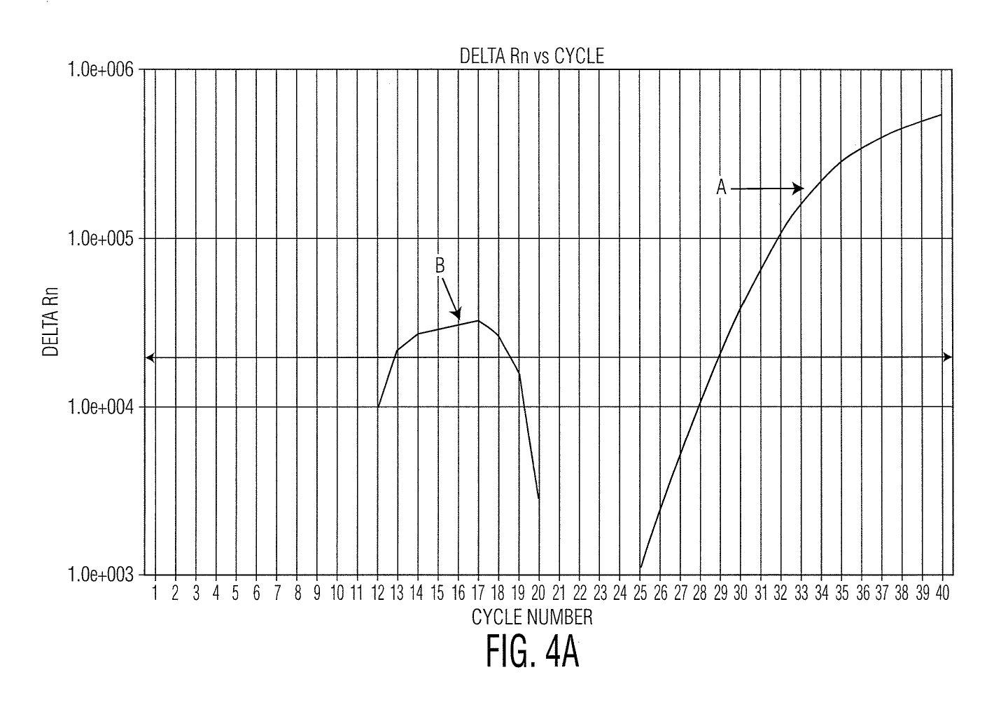

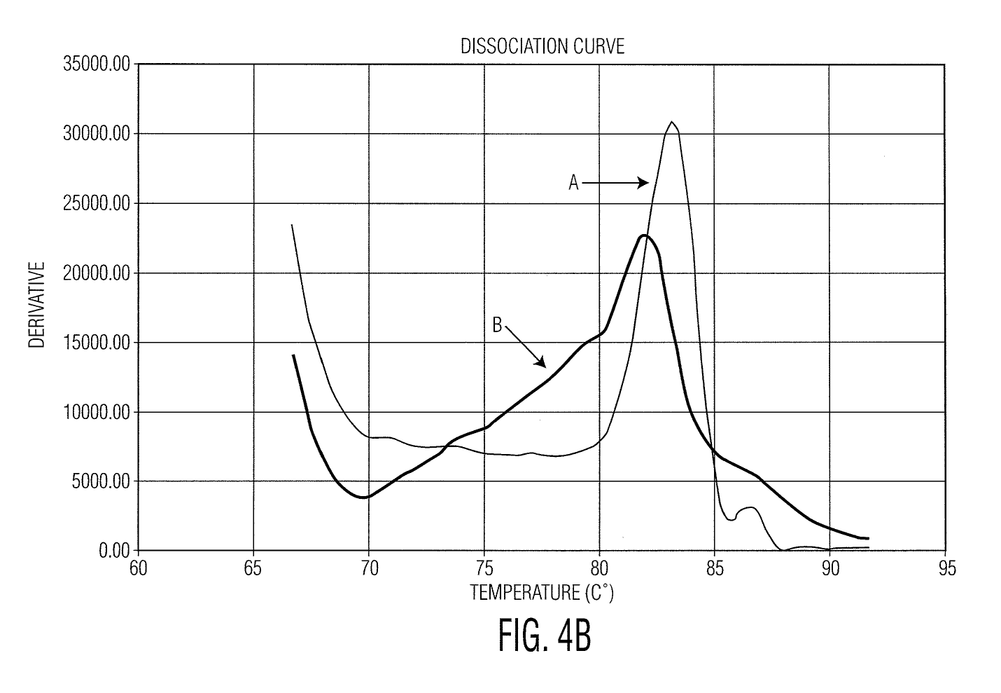

TB specific nucleic acids were detected in a cell-free fraction of pleural effusion of an individual clinically diagnosed with TB (FIG. 4A, arrow A), but not in the sediment fraction of the same pleural effusion sample (FIG. 4A, arrow B). In addition, the sediment fraction show strong non-specific PCR products (FIG. 4B, arrow B).

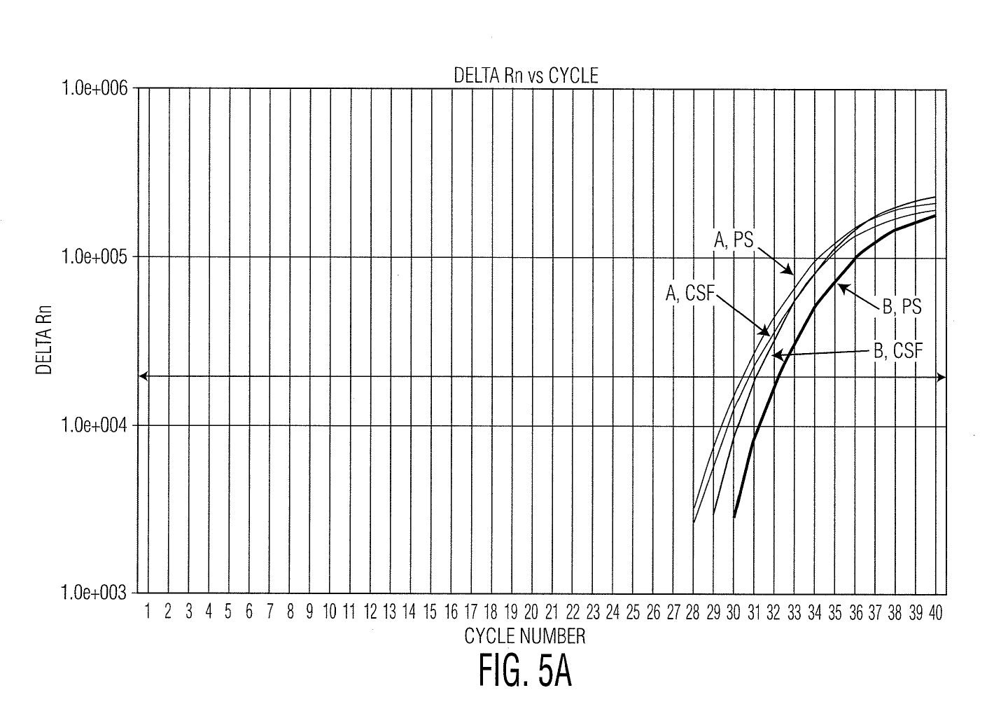

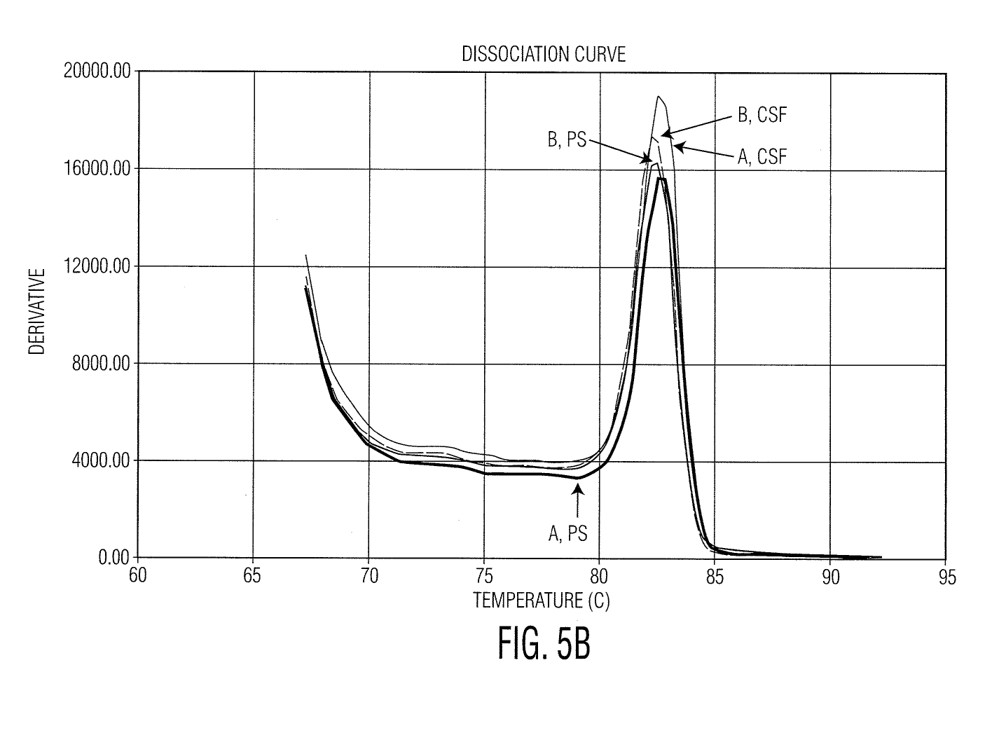

Cell-free fractions of PS and CSF samples from two individuals, A and B, who were clinically diagnosed with TB were analyzed. FIG. 5A shows the comparable levels of TB-derived DNA fragments detected in the cell-free fractions (PS vs, CSF) from individuals A and B. FIG. 5B shows the specific melting peaks of the IS6110 amplicon of TB DNA fragments, indicating no non-specific PCR products.

The detection results using qPCR to detect cell-free TB specific nucleic acid were compared with the TB clinical diagnosis (Table 2), and showed a sensitivity of about 91% (67/74) and a specificity of about 83% (15/18).

TABLE 2
Cell free NA qPCR vs Clinic Diagnosis

Clinical Diagnosis

| | | + | − | Total |
|---|---|---|---|---|
| PCR | + | 67 | 3 | 70 |
| | − | 7 | 15 | 22 |
| | Total | 74 | 18 | 92 |

The target TB specific nucleic acid was quantified. A sample having a Ct value greater than 40 was considered as having 0 copy of the target TB specific nucleic acid. A sample having a Ct of 36-40 was considered to have one copy of the target TB specific nucleic acid.

For a sample having a Ct less than 36, the copy number of the target TB specific nucleic acid was determined using a standard curve as described in Example 1. Among the 67 individuals clinically diagnosed with TB and tested positive with the target TB specific nucleic acid, the average copy number of the target TB specific nucleic acid was 242.6±531.8 per ml of the fraction, Among the 3 individuals not clinically diagnosed for TB, but tested positive with the target TB specific nucleic acid, the average copy number of the target TB specific nucleic acid was 16.2±16.2 per ml of the fraction

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A method for detecting a target nucleic acid derived from a pathogen in a subject, comprising
(a) amplifying the nucleic acid sequence of the target nucleic acid, wherein the target nucleic acid is obtained from a cell-free fraction of a blood sample from the subject, and whereby a double stranded DNA is produced, and
(b) detecting the double stranded DNA, wherein the presence of the double stranded DNA indicates the presence of the target nucleic acid in the subject.

2. The method of claim 1, wherein the target nucleic acid is DNA.

3. The method of claim 1, wherein the target nucleic acid is RNA.

4. The method of claim 1, wherein the cell-free fraction is blood serum.

5. The method of claim 1, wherein the cell-free fraction is blood plasma.

6. The method of claim 1, wherein the pathogen is Mycobacterium Tuberculosis (TB).

7. The method of claim 1, wherein the nucleic acid sequence is derived from a DNA sequence of Mycobacterium Tuberculosis (TB) H37Rv selected from the group consisting of IS6110, IS1084, MPT 64, rrs, esat6, esat6-like, MDR, rpoB, katG, iniB and fragments thereof.

8. The method of claim 1, wherein the double stranded DNA has 40-60 bp.

9. The method of claim 1, wherein the volume of the blood sample is 0.2-10 ml.

10. The method of claim 1, wherein the nucleic acid sequence is amplified by polymer chain reaction (PCR).

11. The method of claim 1, wherein the double stranded DNA is detected by a detecting agent selected from the group consisting of a fluorescence labeled probe, an intercalating fluorescence dye and a primer of Light Upon Extension (LUX).

12. The method of claim 11, wherein the intercalating fluorescence dye is selected from the group consisting of SYBR green, CytoGreen, Eva Green, BOXTO and SYTO9.

13. The method of claim 1, further comprising concentrating the target nucleic acid in the cell-free fraction.

14. The method of claim 1, further comprising preparing the cell-free fraction from the blood sample.

15. The method of claim 1, further comprising diagnosing TB infection in the subject.

16. The method of claim 15, wherein the TB infection is active.

17. The method of claim 15, wherein the TB infection is latent.

18. A kit for detecting a target nucleic acid derived from a pathogen in a subject, comprising
(a) one or more reagents or materials for amplifying the nucleic acid sequence of the target nucleic acid obtained from a cell-free fraction of a blood sample from the subject to produce a double stranded DNA, and
(b) one or more reagents or materials for detecting the double stranded DNA.

19. The kit of claim 18, wherein the one or more reagents or materials for amplifying the target nucleic acid sequence comprise a pair of primers, wherein the double stranded DNA has 40-60 nucleotides.

20. The kit of claim 18, wherein the pathogen is Mycobacterium Tuberculosis (TB).

21. The kit of claim 18, wherein the nucleic acid sequence is derived from a DNA sequence of Mycobacterium Tuberculosis (TB) H37Rv selected from the group consisting of IS6110, IS1084, MPT 64, rrs, esat6, esat6-like, MDR, rpoB, katG, iniB and fragments thereof.

22. The kit of claim 18, wherein the one or more reagents or materials for detecting the double stranded DNA comprises an intercalating fluorescence dye.
